(19) United States
(12) Patent Application Publication
(10) Pub. No.: US 2025/0339641 A1
(43) Pub. Date: Nov. 6, 2025

(54) VALVE ARRANGEMENT FOR A RESPIRATORY MASK

(71) Applicant: Dräger Safety AG & Co. KGaA, Lübeck (DE)

(72) Inventors: Dario DENZLER, Volketswil (CH); Mark DENZLER, Volketswil (CH); Adrian EUGSTER, Bauma (CH)

(21) Appl. No.: 18/869,991

(22) PCT Filed: Feb. 8, 2023
(86) PCT No.: PCT/DE2023/100100

§ 371 Date: Nov. 27, 2024

(30) Foreign Application Priority Data
May 31, 2022 (DE) 10 2022 113 700.7

(51) Int. Cl. A61M 16/20

(52) U.S. Cl. CPC A61M 16/208

(57) ABSTRACT

A valve assembly (101) includes a valve disk (3) for a gas mask (respiratory mask). The valve disk (3) is configured with holding elements (5), which are configured in the form of spring elements (7). The spring elements (7) bring about an inoperative position (8) of the valve disk in the valve assembly (101) in case of an incoming flow from a blocking direction (13). The spring elements (7) bring about a raising of the valve disk (3) into an operating position (10) in case of an incoming flow from a flow direction (15), so that an opening state (9) of the valve assembly (101) is obtained.

### CROSS REFERENCE TO RELATED APPLICATIONS

This application is a United States National Phase Application of International Application PCT/DE2023/100100, filed Feb. 8, 2023, and claims the benefit of priority under 35 U.S.C. § 119 of German Application 102022113700.7, filed May 31, 2022, the entire contents of which are incorporated herein by reference.

### TECHNICAL FIELD

The present invention pertains to configurations of gas masks (respiratory masks). Gas masks are used for protection against harmful gases or substances in the surroundings of people in the widest variety of areas. For example, gas masks are used in areas with elevated dust pollution. Other areas of application are in the clinical setting, for example, as part of protective clothing in areas with infectious patients.

### BACKGROUND

Such masks are also called “FFP” masks, and “FFP” stands for “filtering face piece.” These FFP gas masks can protect against dust and viruses and can be divided into FFP gas masks in different filter protection classes FFP1, FFP2, FFP3. This division is carried out on the basis of a factor of the so-called workplace limit value (WLV). If the allowable workplace limit value is exceeded fourfold, then the wearing of an FFP1 mask is recommended. If the workplace limit value is exceeded tenfold, the wearing of an FFP2 mask is recommended. The wearing of an FFP3 mask is recommended in case of a 30-fold exceeding of the workplace limit value.

There are gas masks in configurations with an exhalation valve or without an exhalation valve.

WO 16146324 A1, EP 1647310 B1 and EP 1224011 B1 show gas masks with an exhalation valve.

U.S. Pat. No. 7,188,622 BB, EP 3675969 A1 and EP 2570156 B1 show exhalation valves for gas masks.

EP 2570156 B1 shows a directional valve with a fixing lug which is arranged off-center to the valve diaphragm and arranged at the valve housing.

US 2008178884 AA shows a valve with a central fastening of the valve diaphragm at a holding structure with a Y-shaped configuration.

U.S. Pat. No. 3,990,439 A shows a valve with a configuration of a holding structure that is formed on one side, i.e., arranged off-center from the center of the valve disk or valve diaphragm for a fastening of the valve disk or valve diaphragm.

Configurations with fastenings of the valve diaphragm, which [fastenings] are arranged centrally in the outflow area of the valve, reduce the opening diameter of the valve that is freely available for the flow. This reduction in the opening diameter largely determines the exhalation resistance of the valve. When the closing action of the valve disk is also caused by means of a gravity action, then a position dependence may occur in the closing action. Such a position dependence may lead to leaks of the gas mask during the use of the gas mask in case of head movements of the respirator user and as a result thereof may then also lead to an impairment of the filtering and protective function of the gas mask. In the further development of exhalation valves for gas masks, there was and is an effort to keep the resistance as low as possible during the exhalation of the user of the gas mask while also maintaining a reliable filtering and protective function.

### SUMMARY

Therefore, an object of the present invention is to provide a valve assembly for a gas mask with a resistance that is as low as possible during exhalation.

Another object of the present invention is to provide a process for the configuration of a valve disk with integral holding elements.

The object is accomplished according to the present invention by a valve assembly comprising a housing, and a valve disk. The holding elements are arranged at the valve disk as integral elements of the valve disk. The valve disk and the holding elements have an essentially planar configuration in at least one state. The holding elements are configured as pretensioned spring elements. The spring elements exert an action of force on the housing by means of the pretension in case of an incoming flow from a blocking direction, so that the valve disk occupies an inoperative position and the valve assembly occupies a first state, in which a flow-through in the blocking direction is prevented. The spring elements are raised from an inoperative position into a second state of an operating position in case of an incoming flow from a flow direction located opposite the blocking direction. The action of force on the housing is reduced, a flow-through of the valve assembly in the flow direction is made possible, and an opening state for the valve assembly is obtained (in the second state operating position).

The object is accomplished according to the present invention by a process for the configuration of a valve disk with integral holding elements with the process comprising configuring a valve disk with integral holding elements, wherein a number of notches (incisions) are formed in a planar valve disk, the notches have predetermined lengths and the notches are configured on two different distances to a center of the valve disk. The notches have an offset, overlapping configuration and the notches form segments in the valve disk. A width of the segments is formed by the two different distances to the center of the valve disk. The segments thus form the integral holding elements of the valve disk as spring elements.

Advantageous embodiments of the present invention appear from this disclosure and are explained in more detail in the following description with partial reference to the figures.

The valve assembly according to the present invention has a low resistance in a flow direction. The valve assembly according to the present invention is closed against the direction of flow, i.e., in a blocking direction. According to the present invention, the valve assembly has a housing and a valve disk. According to the present invention, pretensioned spring elements as holding elements are intended to be integral elements of the valve disk.

The spring elements may also be called spring-loaded arms, wherein the term “spring-loaded arms” also comprises the function of raising the valve disk in a linguistically figuratively understandable manner. The spring elements exert an action of force on the housing or on a sealing contour in case of an incoming flow from a blocking direction by means of the pretension, so that the valve disk occupies an inoperative position and the valve assembly occupies a first state, in which a flow through the valve assembly in the blocking direction is prevented. The spring elements are raised from the inoperative position into a second state of an operating position in case of an incoming flow from a flow direction located opposite the blocking direction, wherein the action of force on the sealing contour is reduced, a flow through the valve assembly in the flow direction is made possible, and an opening state for the valve assembly (101, 201) is obtained. At least two spring elements as integral holding elements are necessary for the embodiment of the function and are embodied by the shape of the valve disk. Practical embodiments have three to six spring elements; however, embodiments with a number of up to 12 spring elements can be constructively configured as well.

In a preferred embodiment of a geometry of a circular or round valve disk, the spring elements are produced, for example, by a number of at least two arc-shaped notches with predetermined lengths being made in the valve disk. The notches with predetermined lengths are provided at two different distances to a center of the valve disk. The notches on the two different distances to the center are configured as being offset to one another. The notches form segments or circular arc sections on the valve disk. The width of the segments is in this case formed by the distance of the two different, spaced-apart distances or diameters to one another, which are defined in relation to the center of the valve disk.

The positions with notches and segments in relation to a center or center of gravity of the selected geometric shape of the valve disk appear in embodiments in which the contour of the valve disk has a configuration deviating from the circular shape. Possible geometric shapes of the valve disk are, for example, elliptical or oval shapes, as well as triangular, square, pentagonal or polygonal shapes. For example, an octagon shape or a nonagon shape may thus be considered to be equivalent to a circular geometry, especially in a configuration with rounded edges, in regard to the arrangement of the segments, so that the properties and advantages of valve disks with round or circular geometry can also be extrapolated to geometries of the valve disk which have a polygonal configuration, and vice versa. Segments in the valve disk appear due to the notches in the valve disk. These segments form holding elements and make possible a degree of freedom of motion in case of a fixed connection or fixing of the valve disk at the center or at the edge or at the outer circumference of the valve disk, so that the valve disk can be raised due to an incoming flow, wherein the segments line up in the form of spring elements and form the integral holding elements of the valve disk as spring elements in this way.

In one preferred embodiment, the spring elements are arranged at an outer area of the valve disk and are connected to the valve assembly. A flow through the valve assembly in the flow direction at the outer area of the valve assembly makes an outflow of exhaled air in this preferred embodiment possible.

In another preferred embodiment, the spring elements are arranged at an inner area of the valve disk and are connected to the valve assembly. The spring elements are in this case arranged preferably symmetrically to a center of the valve disk.

The valve disk is configured in an essentially circular or round shape or an elliptical or oval shape in another preferred embodiment. Elliptical or oval shapes may have advantages with regard to wearing comfort in case of an integration of the valve assembly with the valve disk into a mask body, for example, into a mask body of a textile mask.

In another preferred embodiment, the valve disk has a triangular, square or polygonal configuration. Polygonal shapes may have advantages in the manufacture of the valve disks and in the automated application of the valve disks into a mask body as well.

The spring elements have a Z-shaped or angular configuration in another preferred embodiment. Z-shaped or angular spring elements provide defined dimensions and thus reproducible spring actions for a defined setting of the inoperative position and the operating position of the valve disk.

In another preferred embodiment, the spring elements have a spiral-shaped configuration. Spring elements with a spiral-shaped configuration may have a degressive or progressive function in case of the setting of the inoperative position or operating position of the valve disk due to the manner of the formation of the spirals, for example, spiral-shaped from the center outwards, expanding at a distance from spiral revolution to spiral revolution, or, for example, spiral-shaped from the center outwards, becoming narrower at a distance from spiral revolution to spiral revolution,

Other preferred embodiments specify how the valve disk may be composed of a variety of materials or combinations of materials, and the valve disk may thus be composed of a metallic material, an elastomer material, a plastic material, textile material or a fiber composite. The notches for forming the spring elements may in this case be made, for example, by means of laser cutting, knife cutting or by press cutting.

A method according to the present invention for configuration of a valve disk with integral holding elements is characterized in that
a number of notches is inserted into a planar valve disk,
the notches have predetermined lengths,
the notches are formed on two different distances to a center of the valve disk,
the notches have an offset, overlapping configuration,
   wherein the notches form segments,
   wherein the width of the segments are formed by the two different distances to the center of the valve disk,
      and the segments thus form integral holding elements of the valve disk as spring elements.

In one preferred embodiment of the method with configuration of a round or circular valve disk, the notches have an arc-shaped configuration. In such a configuration of a round or circular valve disk, the segments form circular arc sections.

The present invention will now be explained in more detail by means of the figures below and the corresponding figure descriptions without limitations of the general idea of the present invention. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of the valve disk;

FIG. 2 is a perspective view of the valve disk according to FIG. 1, shown in an opening state;

FIG. 3 is a schematic view showing an alternative configuration of valve disk;

FIG. 4 is a schematic view showing an alternative configuration of valve disk;

FIG. 5 is a schematic view showing an alternative configuration of valve disk;

FIG. 6 is a schematic view showing an alternative configuration of valve disk;

FIG. 7 is a schematic partially sectional view of a valve assembly, and

FIG. 8 is a schematic partially sectional view of an alternative valve assembly.

### DESCRIPTION OF PREFERRED EMBODIMENTS

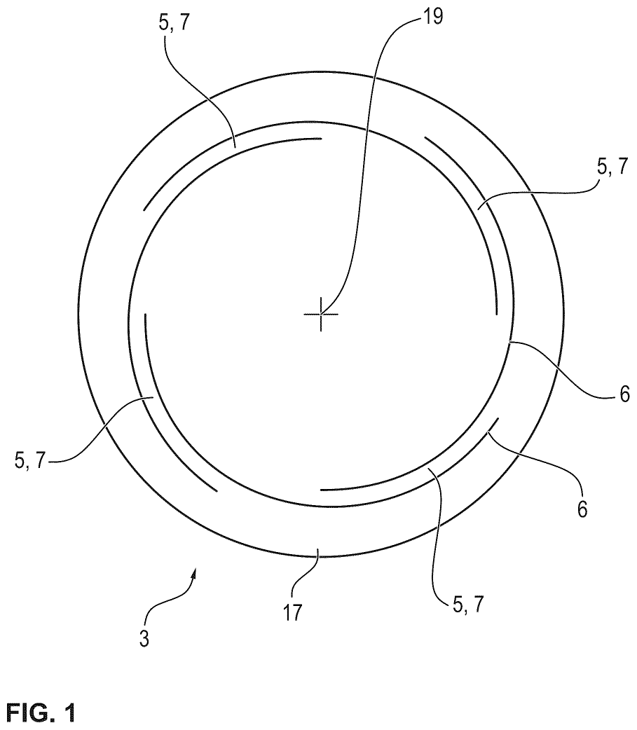

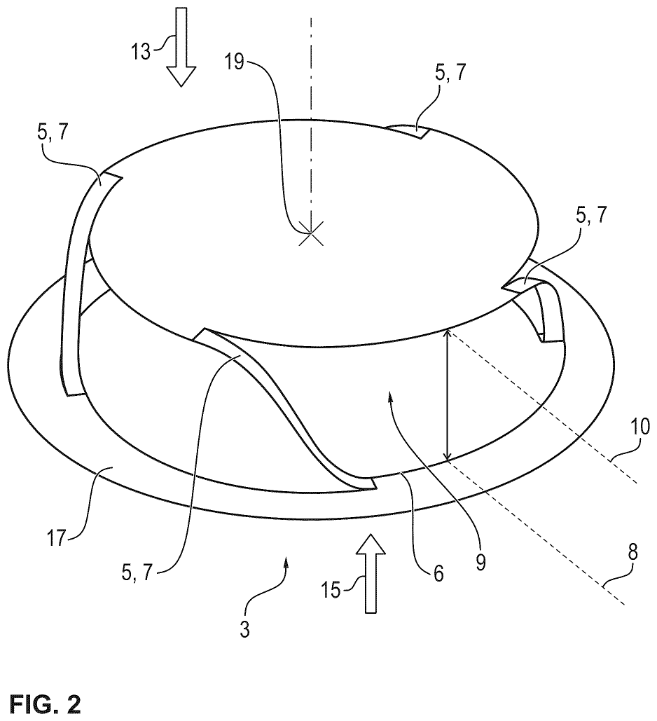

Referring to the drawings, FIG. 1 shows a schematic view of a valve disk 3 with holding elements 5 configured in the form of spring elements 7. The valve disk 3 is shown with the center 19 and the edge area 17. Notches 6 which are formed on different distances to the center 19 are shown. The notches 6 form the spring elements 7 as segments of the valve disk 3, which make possible a change in position of the valve disk 3 from an inoperative position 8 (FIG. 2) into an operating position 10 (FIG. 2). A number of four spring elements 7 are formed in this FIG. 1 by a total number of eight notches.

FIG. 2 shows the valve disk 3 according to FIG. 1 in a perspective view in an opening state 9, in which the valve disk 3 is raised due to a flow from a flow direction 15 and held in a raised position 10. Identical elements in FIG. 1 and in FIG. 2 are designated with the same reference numbers in FIGS. 1 and 2. The raised position 10 is defined by the holding elements 5 configured as spring elements 7. In the raised position 10, the valve disk is raised at least with considerable area portions about the center 19. Quantities of gas may flow upwards from the flow direction 15 and then may flow out or continue to flow at the circumferential area 17 of the valve disk 3.

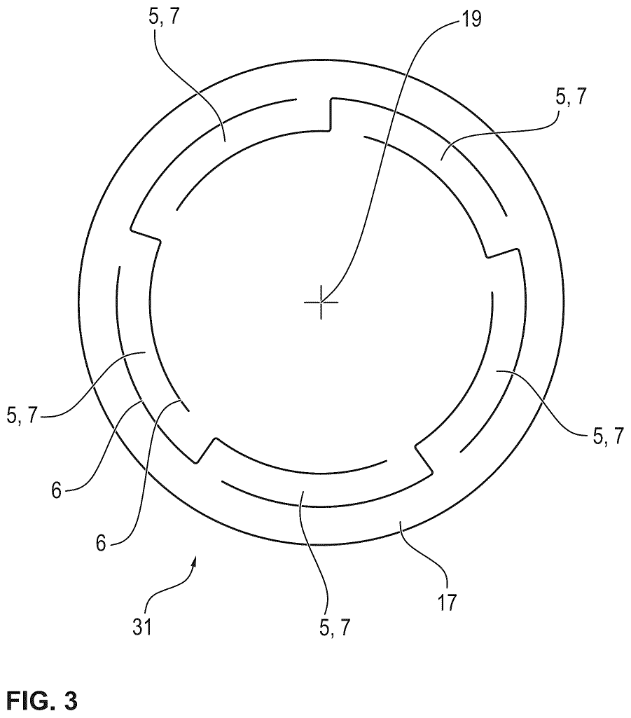

FIGS. 3 through 6 schematically show various other configurations of planar valve disks. Identical elements in FIGS. 1 through 6 are designated with the same reference numbers in FIGS. 1 through 6. FIG. 3 shows a valve disk 31 with notches 6, which produces a configuration of the spring elements 7 in a Z-shaped or angular shape. In this FIG. 3, a number of five spring elements 7 are formed in the valve disk 31 due to a total number of five angular-shaped notches 6.

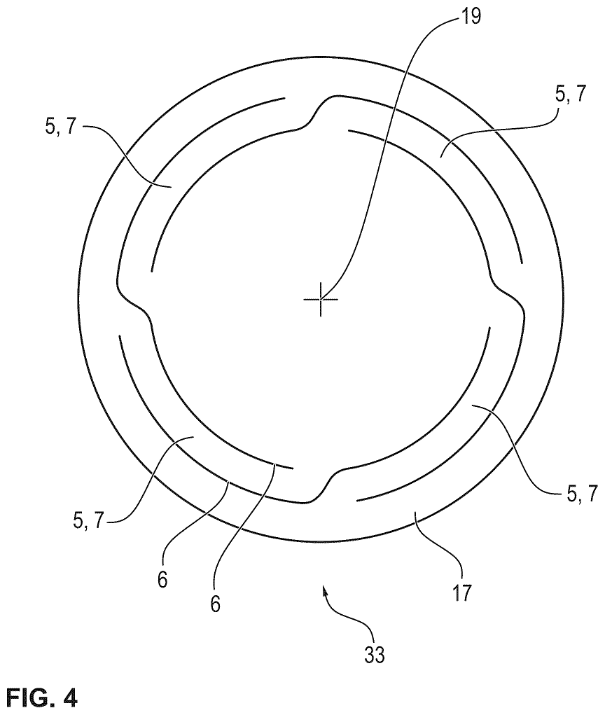

FIG. 4 shows a valve disk 31 with notches 6, which produces a configuration of the spring elements 7 in a rounded variant of a Z-shaped or angular shape. In this FIG. 4, a number of four spring elements 7 in the valve disk 33 are formed by a total number of four notches 6 with a rounded, angular shape.

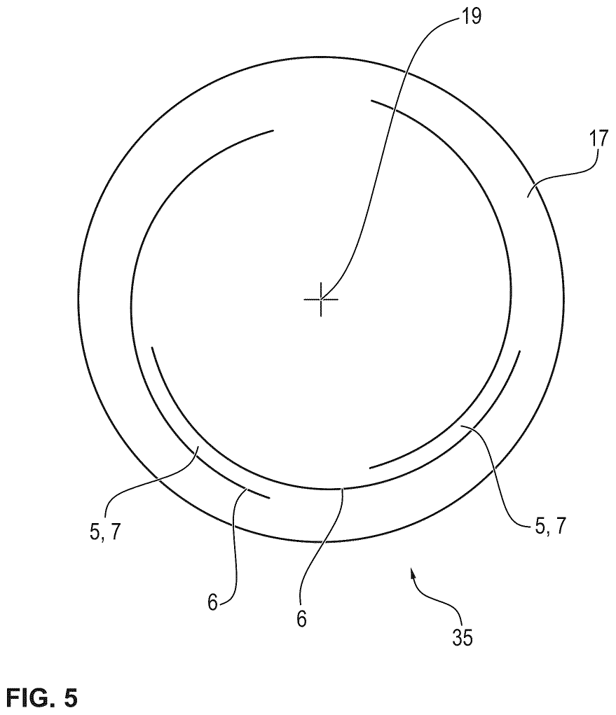

FIG. 5 shows a valve disk 35 with three notches 6, which produces a configuration of the spring elements 7 in an asymmetrical distribution of the spring elements 7 at the circumference 17 of the valve disk 35.

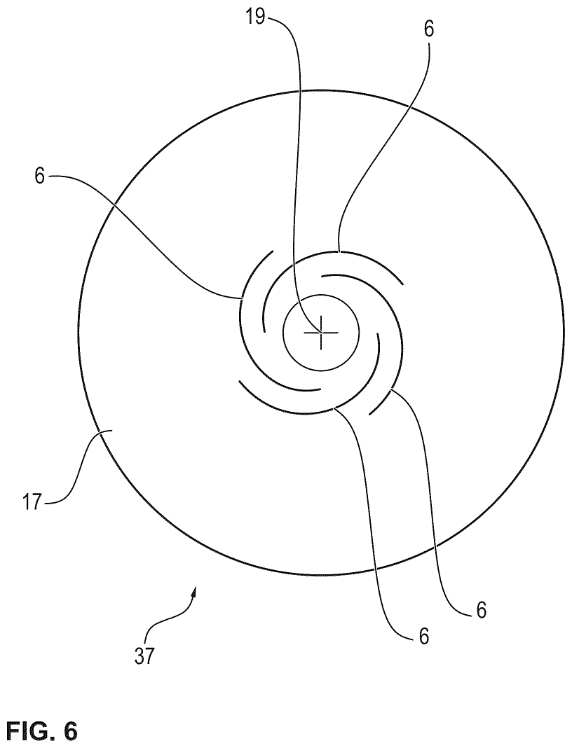

FIG. 6 shows a valve disk 36 with four notches 6, which produces a configuration of the spring elements 7 symmetrical to and close to the center 19 of the valve disk 37.

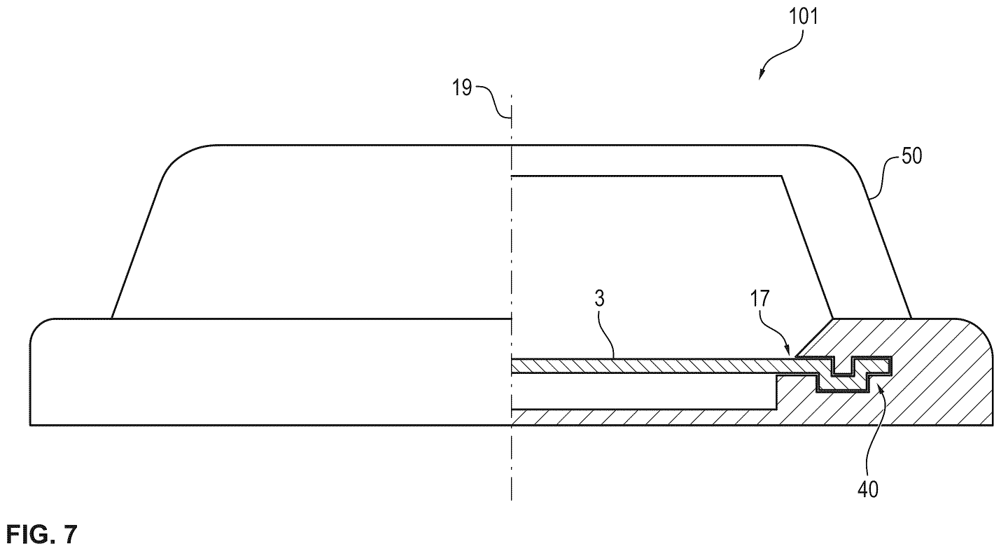

FIG. 7 shows a schematic view 100 of a valve assembly 101 in a configuration for use with planar valve disks according to FIGS. 1 through 5 in a housing 50. The planar valve disks 3, 31, 33, 35 according to FIGS. 1 through 5 may be arranged in the valve assembly 101 and may be held, fixed or fastened at the edge area 17 by means of a clamping geometry 40. The function of the valve assembly 101—for example, the valve disk 3 according to the views in FIGS. 1 and 2—with changes in position from an inoperative position 8 (FIG. 2) into an operating position 10 (FIG. 2), as explained on the basis of FIGS. 1 and 2.

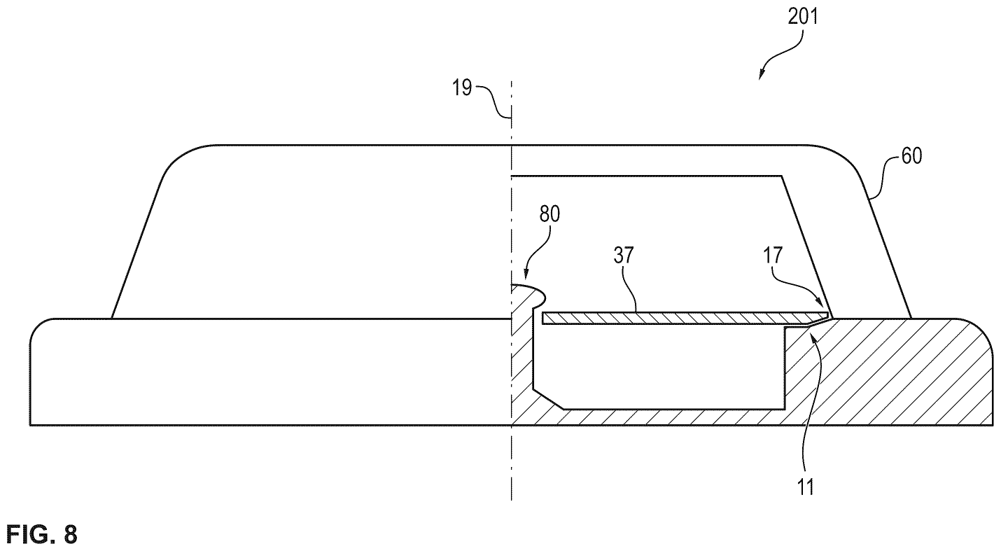

FIG. 8 shows a schematic view 200 of an alternative valve assembly 201 with a planar valve disk 37 according to FIG. 6 in a housing 60. The planar valve disk 37 according to FIG. 6 may be arranged in the valve assembly 201 and be held, fixed or fastened in a floating manner at the center 19 by means of a fastening point 70. In contrast to the function of the valve assembly 101, the function of the valve assembly 201 results in a deviating manner by the fastening and arrangement of the valve disk 37 in the housing 60 being configured as being mounted in a floating manner. The valve disk 37 is located on a valve seat 11 with an edge 17 of the valve disk 37 and seals there in an inoperative position. The valve seat 11 is part of the housing 60.

While specific embodiments of the invention have been shown and described in detail to illustrate the application of the principles of the invention, it will be understood that the invention may be embodied otherwise without departing from such principles.

### LIST OF REFERENCE NUMBERS

3 Valve disk
5 Holding elements
6 Notches
7 Spring elements
8 Inoperative position
9 Opening state
10 Operating position
11 Sealing contour
13 Blocking direction
15 Flow direction
17 Outer area, edge/circumferential area of the valve disk
18 Inner area, middle area of the valve disk
19 Center of the valve disk
31, 33, 35, 37 Valve disk
40 Clamping geometry
50, 60 Housing
70 Fastening point
80 Floating mounting

1. A valve assembly comprising:
a housing;
a valve disk; and
holding elements arranged at the valve disk as integral elements of the valve disk,
wherein the valve disk and the holding elements have an essentially planar configuration,
wherein the holding elements are configured as pretensioned spring elements wherein the spring elements have a spiral-shaped configuration, and the spring elements exert an action of force on the housing by means of pretension in case of an incoming flow from a blocking direction, so that the valve disk occupies an inoperative position and the valve assembly occupies a first state, in which a flow-through in the blocking direction is prevented,
wherein the spring elements are raised from an inoperative position into a second state of an operating position in case of an incoming flow from a flow direction located opposite the blocking direction, wherein the action of force on the housing is reduced, a flow-through of the valve assembly in the flow direction is made possible, and an opening state for the valve assembly is obtained.

2. A valve assembly in accordance with claim 1, wherein the spring elements are arranged at an outer area of the valve disk and are connected to the valve assembly.

3. A valve assembly in accordance with claim 1, wherein the spring elements are arranged at an inner area of the valve disk and are connected to the valve assembly.

4. A valve assembly in accordance with claim 1, wherein the valve disk is configured in an essentially circular or round shape or an elliptical or oval shape.

5. A valve assembly in accordance with claim 1, wherein the valve disk has a triangular-shaped, square-shaped or polygonal-shaped configuration.

6. A valve assembly in accordance with claim 1, wherein the spring elements have a Z-shaped or angular-shaped configuration.

7. (canceled)

8. A valve assembly in accordance with claim 1, wherein the valve disk is composed of a metallic material, an elastomer material, a plastic material, a textile material or a fiber composite.

9. A process for configuring a valve disk with integral holding elements,
wherein a number of notches are formed in a planar valve disk,
wherein the notches have predetermined lengths,
wherein the notches are configured on two different distances to a center of the valve disk,
wherein the notches overlap with an offset configuration,
wherein the notches form segments in the valve disk,
wherein the width of the segments is formed by the two different distances to the center of the valve disk, and the segments thus form the integral holding elements of the valve disk as spring elements.

10. A process in accordance with claim 9, wherein the notches are formed in the valve disk such that spring elements have a Z-shaped, angular-shaped or spiral-shaped configuration.

11. A valve assembly comprising:
a housing;
a valve disk with an essentially planer configuration; and
holding elements arranged at the valve disk and configured in one piece with the valve disk, the holding elements having an essentially planar configuration with the valve disk in an inoperative position and with the valve assembly in a first state and having a spiral-shaped configuration with the valve disk in an operative position and with the valve assembly in a second state, the holding elements being configured as pretensioned spring elements that exert an action of force between the valve disk and the housing by means of pretension,
wherein in case of an incoming flow from a blocking direction, the valve disk occupies the inoperative position and the valve assembly occupies the first state, in which a flow-through in the blocking direction is prevented,
wherein in case of an incoming flow from a flow direction, located opposite the blocking direction, the spring elements are raised from the inoperative position into the operative position, with the valve assembly in the second state, whereby the action of force between the valve disk and the housing is reduced and a flow-through of the valve assembly in the flow direction is made possible, and an open state of the valve assembly is obtained.

12. A valve assembly in accordance with claim 11, wherein the spring elements are arranged at an outer area of the valve disk and are connected to the housing.

13. A valve assembly in accordance with claim 11, wherein the spring elements are arranged at an inner area of the valve disk and are connected to the housing.

14. A valve assembly in accordance with claim 11, wherein the valve disk is configured in an essentially circular or round shape or an elliptical or oval shape.

15. A valve assembly in accordance with claim 11, wherein the valve disk has a triangular-shaped, square-shaped or polygonal-shaped configuration.

16. A valve assembly in accordance with claim 11, wherein the spring elements have a Z-shaped or angular-shaped configuration in the operative position.

17. A valve assembly in accordance with claim 11, wherein the valve disk is comprised of a metallic material, an elastomer material, a plastic material, a textile material or a fiber composite.
